(19) United States
(12) Patent Application Publication
(10) Pub. No.: US 2019/0103194 A1
(43) Pub. Date: Apr. 4, 2019

(54) HEALTHCARE SYSTEM THAT FACILITATES PATIENT-CUSTOMIZED HEALTHCARE SERVICES FROM MULTIPLE HEALTHCARE ORGANIZATIONS VIA A SINGLE HEALTHCARE APPLICATION

(71) Applicant: Practive Health Inc., Raleigh, NC (US)

(72) Inventors: Pradeep Ramayya, Glasgow (GB); Anjali Ramayya, Glasgow (GB); Dean Burton, Johnstone (GB); Alan M. Pinder, Helensburgh (GB); Suresh Mateti, Hyderabad (IN)

(21) Appl. No.: 16/151,802

(22) Filed: Oct. 4, 2018

Related U.S. Application Data
(60) Provisional application No. 62/568,137, filed on Oct. 4, 2017.

(51) Int. Cl. G16H 80/00; G16H 10/60; G16H 20/70; H04W 12/06; G09B 5/06

(52) U.S. Cl. CPC G16H 80/00; G16H 10/60; G06F 3/0481; H04W 12/06; G09B 5/065; G16H 20/70

(57) ABSTRACT

A healthcare system that facilitates patient-customized healthcare services from multiple healthcare organizations via a single healthcare application is disclosed. A mobile computing device authenticates a patient with a first healthcare server application associated with a first healthcare organization. The mobile computing device receives from the first healthcare server application a plurality of healthcare organization (HCO) pages, at least some of the plurality of HCO pages comprising information specific to a first medical condition of the patient. The mobile computing device generates a user interface based on a plurality of common user interface components maintained in the mobile computing device and the first plurality of HCO pages, and presents, on a display device of the mobile computing device, the first user interface, the first user interface including the plurality of common user interface components and at least some of the information specific to the first medical condition of the patient.

### RELATED APPLICATIONS

This application claims the benefit of U.S. Provisional Application No. 62/568,137, filed on Oct. 4, 2017, entitled “MOBILE HEALTH APPLICATION,” the disclosure of which is hereby incorporated herein by reference in its entirety.

### TECHNICAL FIELD

The embodiments relate generally to healthcare systems, and in particular to a healthcare system that facilitates patient-customized healthcare services from multiple healthcare organizations via a single healthcare application.

### BACKGROUND

It is not uncommon for an individual to utilize the services of multiple different healthcare organizations that each address a different healthcare condition or set of conditions. The term “healthcare organization” (HCO) is used herein to refer to any entity that provides treatment or other services in the healthcare industry. It is increasingly common for an HCO to provide information and services electronically over the Internet to a user's desktop or mobile computing device. However, each HCO typically offers a completely different healthcare application that offers the user a completely different user interface, and that has a completely different look and feel. The user must therefore master, for each different HCO, the particular healthcare application associated with the respective HCO, including having to learn where in the healthcare application different types of information is located, how to communicate electronically with the HCO, and the like. This can inhibit the effectiveness of such healthcare applications, and potentially keep users from using them at all, thus preventing users from the healthcare benefits provided by such healthcare applications. Moreover, the need to learn multiple different healthcare applications can be daunting for many users, and in particular for users who have not spent a lifetime working with computing devices.

### SUMMARY

The embodiments include a healthcare system that facilitates patient-customized healthcare services from multiple healthcare organizations via a single healthcare application. The healthcare application includes a non-customized framework, and is configured to receive customized information from healthcare server applications associated with corresponding healthcare organizations as well as other external sources. Among other advantages, the healthcare application generates, from the non-customized framework of the healthcare application and the customized information from a healthcare server application, a customized user interface that has common look and feel components with customized information based on the particular medical conditions and care plan associated with the patient.

Those skilled in the art will appreciate the scope of the disclosure and realize additional aspects thereof after reading the following detailed description of the embodiments in association with the accompanying drawing figures.

### BRIEF DESCRIPTION OF THE DRAWINGS

The accompanying drawing figures incorporated in and forming a part of this specification illustrate several aspects of the disclosure and, together with the description, serve to explain the principles of the disclosure.

FIG. 1 is a block diagram of an environment in which embodiments can be practiced;

FIG. 2 is a block diagram of the environment illustrated in FIG. 1 at a point in time subsequent to that of FIG. 1;

FIG. 3 is a flowchart of a method for facilitating patient-customized healthcare services from multiple healthcare organizations via a single healthcare application according to one embodiment

FIG. 4 is a block diagram illustrating additional aspects of the healthcare system according to some embodiments;

FIG. 5 is a block diagram of a portion of the environment suitable for illustrating additional aspects of the healthcare system disclosed herein according to some embodiments.

FIG. 6 is a diagram illustrating how a healthcare server application (HCSA) may update user information of a patient in response to the receipt of new information associated with the patient according to one embodiment;

FIG. 7 is a diagram illustrating how the HCSA may update the user information of a patient in response to the receipt of new information associated with the patient according to another embodiment; and

FIG. 8 is a block diagram of a computing device suitable for implementing any of the computing devices discussed herein.

### DETAILED DESCRIPTION

The embodiments set forth below represent the information to enable those skilled in the art to practice the embodiments and illustrate the best mode of practicing the embodiments. Upon reading the following description in light of the accompanying drawing figures, those skilled in the art will understand the concepts of the disclosure and will recognize applications of these concepts not particularly addressed herein. It should be understood that these concepts and applications fall within the scope of the disclosure and the accompanying claims.

Any flowcharts discussed herein are necessarily discussed in some sequence for purposes of illustration, but unless otherwise explicitly indicated, the embodiments are not limited to any particular sequence of steps. The use herein of ordinals in conjunction with an element is solely for distinguishing what might otherwise be similar or identical labels, such as “first message” and “second message,” and does not imply a priority, a type, an importance, or other attribute, unless otherwise stated herein. The term “about” used herein in conjunction with a numeric value means any value that is within a range of ten percent greater than or ten percent less than the numeric value.

As used herein and in the claims, the articles “a” and “an” in reference to an element refers to “one or more” of the element unless otherwise explicitly specified.

The embodiments include a healthcare system that facilitates patient-customized healthcare services from multiple healthcare organizations via a single healthcare application. The healthcare application includes a non-customized framework, and is configured to receive customized information from healthcare server applications associated with corresponding healthcare organizations and other external sources. Among other advantages, the healthcare application generates, from the non-customized framework of the healthcare application and the customized information from a healthcare server application, a customized user interface that has common look and feel components with customized information based on the particular medical conditions and care plan associated with the patient.

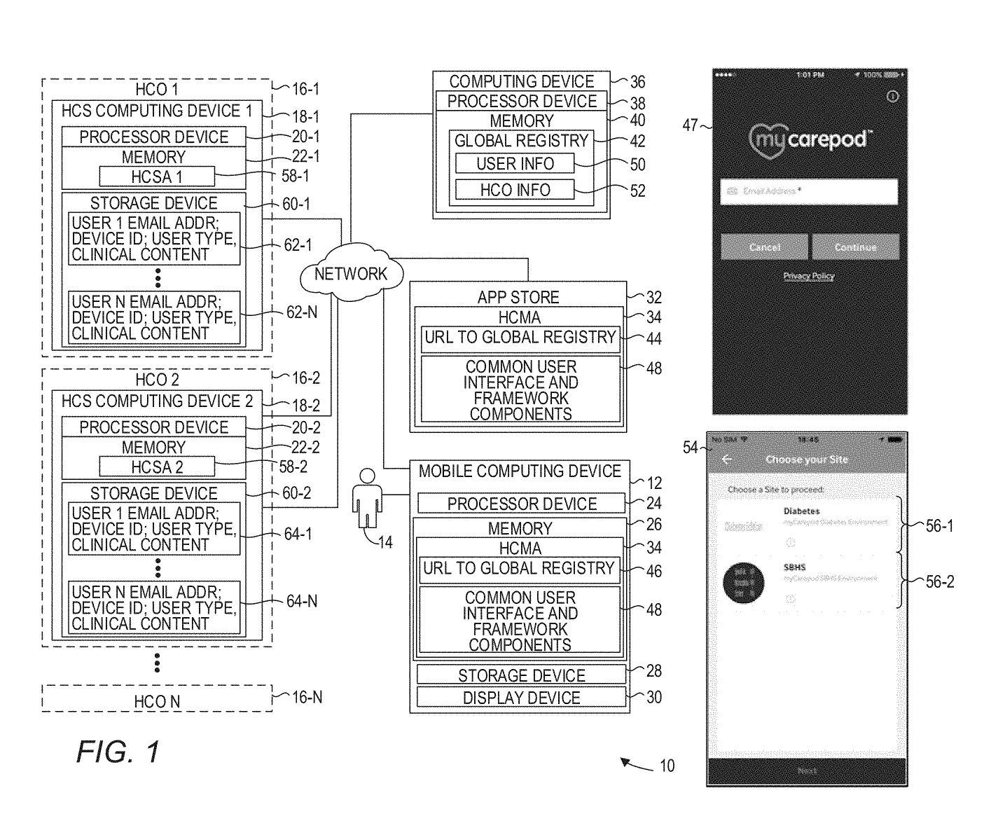

FIG. 1 is a block diagram of an environment 10 in which embodiments can be practiced. The environment 10 includes a mobile computing device 12 associated with a patient 14. The environment 10 also includes one or more healthcare organizations (HCOs) 16-1-16-N (generally, HCOs 16), each of which provides healthcare services to one or more patients. Merely as an example, the HCO 16-1 provides healthcare services relating to the medical condition diabetes, and the HCO 16-2 provides healthcare services relating to the medical condition spina bifida. While for purposes of illustration each HCO 16 is discussed herein as providing healthcare services for only one medical condition, it should be apparent that an HCO 16 may provide healthcare services for any number of medical conditions. Each HCO 16 includes a healthcare server (HCS) computing device. In this example, the HCO 16-1 includes an HCS computing device 18-1, and the HCO 16-2 includes an HCS computing device 18-2. The HCS computing device 18-1 includes a processor device 20-1 and a memory 22-1, and the HCS computing device 18-2 includes a processor device 20-2 and a memory 22-2.

The mobile computing device 12 includes a processor device 24, a memory 26, a storage device 28, and a display device 30. In this example, the patient 14 has diabetes and spina bifida, and is a patient of both the HCO 16-1 and the HCO 16-2. The patient 14 directs the mobile computing device 12 to connect to an application store 32, such as the Apple App Store, the Google Play application store, or any other suitable location from which an application can be downloaded. The patient 14 downloads from the application store 32 onto the mobile computing device 12 a healthcare mobile application (HCMA) 34.

The environment 10 also includes a computing device 36 that has a processor device 38 and a memory 40. The computing device 36 hosts a global registry 42. The HCMA 34 includes an electronic address 44 to the global registry 42. The electronic address 44 may comprise, by way of non-limiting example, a uniform resource locator (URL) or any other suitable identifier of the location of the global registry 42. The HCMA 34 also includes common user interface and framework components 48. When the HCMA 34 is first downloaded onto the mobile computing device 12, the HCMA 34 contains no medical information regarding the patient 14, or any content unique or specific to any HCO 16.

The patient 14 executes the HCMA 34 on the mobile computing device 12. The HCMA 34 presents on the display device 30 a user interface 47 that requests authentication information from the patient 14. The patient 14 enters the authentication information, such as an email address and password, for purposes of authentication with the global registry 42. The HCMA 34 sends the authentication information entered by the patient 14 to the global registry 42. The global registry 42 accesses user information 50 and determines, based on the authentication information, that the patient 14 has entered the proper authentication information. The global registry 42 accesses HCO information 52 and determines that the patient 14 is a patient of both the HCO 16-1 and HCO 16-2. The global registry 42 sends a message to the HCMA 34 that includes an electronic address of the HCS computing device 18-1 associated with the HCO 16-1, and an electronic address of the HCS computing device 18-2 is associated with the HCO 16-2.

The HCMA 34 presents, on the display device 30, a user interface 54 that includes a healthcare organization reference 56-1 that refers to the HCO 16-1, and a healthcare organization reference 56-2 that refers to the HCO 16-2. For purposes of illustration, assume that the patient 14 wishes to receive medical information from the HCO 16-1, and thus selects, such as by touching, the healthcare organization reference 56-1. The HCMA 34 receives the input that selects the healthcare organization reference 56-1, and, using the electronic address of the HCS computing device 18-1 associated with the HCO 16-1 that was received from the global registry 42, passes information that identifies the patient 14 to the HCS computing device 18-1.

The HCS computing device 18-1 includes a healthcare server application (HCSA) 58-1 which, in conjunction with the HCMA 34, make up the healthcare system disclosed herein. As will be discussed in greater detail below, the HCMA 34 initially contains only the common user interface framework components 48, and contains no information about the patient 14 and contains no information unique or specific to the HCO 16-1. The HCSA 58-1 sends customized content to the HCMA 34 relating to the diabetes health condition of the patient 14, and the HCMA 34 integrates the customized content with the common user interface and framework components 48 to provide the patient 14 with medical information and services that are unique to the HCO 16-1, but that are presented to the patient 14 on the display device 30 with a common “look and feel” using the common user interface and framework components 48.

Similarly, the HCS computing device 18-2 includes an HCSA 58-2. Upon proper authentication with the HCMA 34, the HCSA 58-2 sends customized content that includes medical information and services for the health condition spina bifida to the patient 14. While the medical information and services for spina bifida will differ from those relating to diabetes, and although the HCO 16-2 is an independent entity from the HCO 16-1, the HCMA 34 will integrate the medical information and content received from the HCSA 58-2 with the common user interface and framework components 48, and will present such information to the patient 14 on the display device 30. The information will have the same “look and feel” and same user interface components as those used to present medical information and content received from the HCSA 58-1. This common user interface vastly simplifies interacting with the HCO 16-1 and 16-2 by the patient 14, because irrespective of the medical condition, or HCO 16, information is presented in a same manner. Moreover, information sought by either the HCO 16-1 or the HCO 16-2 is similarly requested in a same manner. Thus, the patient 14 need only learn how to interact with the HCMA 34, irrespective of the number of HCOs 16 of which the patient 14 is a patient.

In this example, the HCS computing device 18-1 includes a storage device 60-1 on which a plurality of user information 62-1-62-N (generally, user information 62) is maintained. Each user information 62 includes information, such as, in the case of a patient, medical information relating to the patient. For example, the user information 62-1 contains information about the patient 14. This information may include, for example, authentication information that is suitable for authenticating the patient 14 to the HCS computing device 18-1, information that identifies a user type of the patient 14, such as a patient, a doctor, or a care circle member, and medical data, such as medical history, labs, and the like, relating to the patient 14. After authenticating the patient 14 with the HCS computing device 18-1, the HCSA 58-1 accesses the user information 62-1, and sends certain of the user information 62-1 to the HCMA 34. The HCMA 34 integrates the information received from the HCSA 58-1 with the common user interface and framework components 48 to present a user interface on the display device 30 that contains medical information for the diabetes health condition and that is specific to the patient 14.

Similarly, the HCS computing device 18-2 includes a storage device 60-2 on which a plurality of user information 64-1-64-N (generally, user information 64) is maintained. Each user information 64 includes information, such as, in the case of a patient, medical information relating to the patient. For example, the user information 64-1 contains information about the patient 14. This information may include, for example, authentication information that is suitable for authenticating the patient 14 to the HCS computing device 18-2, information that identifies a user type of the patient 14, such as a patient, a doctor, or a care circle member, and medical data, such as medical history, labs, and the like, relating to the patient 14. After authenticating the patient 14 with the HCS computing device 18-2, the HCSA 58-2 accesses the user information 64-1, and sends certain of the user information 64-1 to the HCMA 34. The HCMA 34 integrates the information received from the HCSA 58-1 with the common user interface and framework components 48 to present a user interface on the display device 30 that contains medical information for the spina bifida health condition and that is specific to the patient 14.

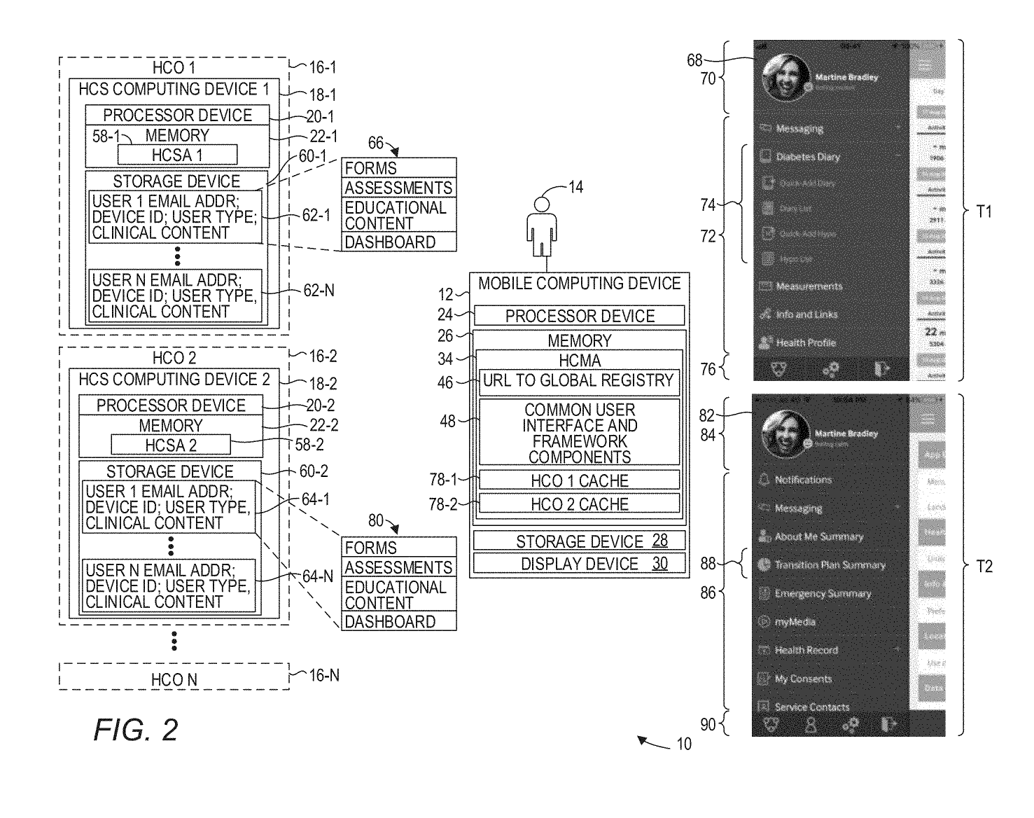

FIG. 2 is a block diagram of the environment 10 at a point in time subsequent to that of FIG. 1. Some of the components illustrated in FIG. 1 have been omitted from FIG. 2 for purposes of illustration. At a time T1, the patient 14 desires to interact with the HCO 16-1. The patient 14 indicates to the HCMA 34 such as via the user interface 54 (FIG. 1) that the patient 14 desires to connect to the HCO 16-1. The HCMA 34 interacts with the HCS computing device 18-1 to authenticate the patient 14 to the HCS computing device 18-1. For purposes of illustration assume that the patient 14 properly authenticates with the HCS computing device 18-1.

The HCS computing device 18-1 accesses the user information 62-1 associated with the patient 14. The user information 62-1 includes clinical content 66 that relates specifically to the patient 14 and/or the particular health condition of diabetes. As an example, the clinical content 66 may include forms that the patient 14 has completed, or needs to complete, assessments that the patient 14 has completed or needs to complete, educational content that may be provided to the patient 14 based on the particular medical condition of diabetes, and/or based on one or more other triggers, as will be discussed in greater detail below. The clinical content 66 may also include a dashboard which comprises a summary of information relating to the patient 14. The HCS computing device 18-1 uses the clinical content 66 to generate a plurality of HCO pages that are customized for the patient 14 and that contain information about the patient 14 and about the medical condition of diabetes. As non-limiting examples, the plurality of HCO pages may include one or more of previous physiological measurements of the patient 14, medications prescribed to the patient 14, laboratory reports of the patient 14, and/or educational materials relating to diabetes. The HCS computing device 18-1 sends the plurality of HCO pages to the HCMA 34. The HCS computing device 18-1 may also send timestamps that correspond to the HCO pages along with the HCO pages to the HCMA 34.

The HCMA 34 receives the plurality of HCO pages and timestamps and generates a user interface 68 based on the common user interface and framework components 48 and the plurality of HCO pages received from the HCS computing device 18-1. The HCMA 34 presents the user interface 68 on the display device 30. The user interface 68, due to the common user interface and framework components 48, will have similar visual characteristics to user interfaces generated by the HCMA 34 irrespective of which HCO 16 the patient 14 is interacting with. For example, the user interface 68 includes an identification portion 70 that contains, in this example, a photograph of the patient 14, a name of the patient 14 in a predetermined font having a predetermined foreground color and predetermined background color. Below the identification portion 70 is a medical information portion 72 that includes a plurality of different selectable rows. Each row is identified by text having a particular font, font size, font color, and is presented on a particular background color. In this example, the medical information portion 72 includes a plurality of rows 74 that relate to the diabetes medical condition of the patient 14. Note that selecting the rows of the medical information portion 72 results in the presentation of customized data obtained from the HCS computing device 18-1 that pertains to the topic of the selected row. A bottom portion 76 contains one or more icons that allow the patient 14 to, for example, access settings of the HCMA 34, access a care circle of support individuals, as will be discussed in greater detail below, upload information via the HCMA 34, and the like.

The HCMA 34 stores the HCO pages received from the HCS computing device 18-1, and the timestamps relating to such pages in an HCO cache 78-1. The HCMA 34 and the HCS computing device 18-1 can then subsequently exchange timestamp information to ascertain if new HCO pages exist on the HCS computing device 18-1 that have not been provided to the HCMA 34. If so, the HCS computing device 18-1 can send the new HCO pages and timestamps that correspond to the new HCO pages to the HCMA 34. In this manner, the HCS computing device 18-1 need not continually resend the same data to the HCMA 34.

Assume that the patient 14 subsequently indicates to the HCMA 34 that the patient wishes to disconnect from the HCS computing device 18-1, such as by logging off the HCMA 34. At a time T2, the patient 14 indicates to the HCMA 34 that the patient 14 desires to connect to the HCO 16-2 to obtain customized information about the spina bifida medical condition of the patient 14. The HCMA 34 interacts with the HCS computing device 18-2 to authenticate the patient 14 to the HCS computing device 18-2. For purposes of illustration assume that the patient 14 properly authenticates with the HCS computing device 18-2.

The HCS computing device 18-2 accesses the user information 64-1 associated with the patient 14. The user information 64-1 also includes clinical content 80 that relates specifically to the patient 14 and/or the particular health condition of spina bifida. As an example, the clinical content 80 may include forms that the patient 14 has completed, or needs to complete, assessments that the patient 14 has completed or needs to complete, educational content that may be provided to the patient 14 based on the particular medical condition of spina bifida, and/or based on one or more other triggers, as will be discussed in greater detail below. The clinical content 80 may also include a dashboard which comprises a summary of information relating to the patient 14. The HCS computing device 18-2 uses the clinical content 80 to generate a plurality of HCO pages that are customized for the patient 14 in that the pages contain information about the patient 14 and about the medical condition of spina bifida. As non-limiting examples, the plurality of HCO pages may include one or more of previous physiological measurements of the patient 14, medications prescribed to the patient 14, laboratory reports of the patient 14, a shunt alert card for the patient 14, and/or educational materials relating to spina bifida. The HCS computing device 18-2 sends the plurality of HCO pages to the HCMA 34. The HCS computing device 18-2 may also send timestamps that correspond to the HCO pages along with the HCO pages to the HCMA 34.

The HCMA 34 receives the plurality of HCO pages and timestamps and generates a user interface 82 based on the common user interface and framework components 48 and the plurality of HCO pages received from the HCS computing device 18-2. The HCMA 34 presents the user interface 82 on the display device 30. The user interface 82, due to the common user interface and framework components 48, has similar visual characteristics to the user interface 68 generated by the HCMA 34 when interacting with the HCS computing device 18-1. For example, the user interface 82 includes an identification portion 84 that contains the photograph of the patient 14, the name of the patient 14 in the predetermined font having the predetermined foreground color and predetermined background color. Below the identification portion 84 is a medical information portion 86 that includes a plurality of different selectable rows. Each row is identified by text having the particular font, font size, font color, and is presented on the particular background color. In this example, the medical information portion 86 includes a row 88 that relates to the spina bifida medical condition of the patient 14. Note that selecting the rows of the medical information portion 86 results in the presentation of customized data obtained from the HCS computing device 18-2 that pertains to the topic of the selected row. A bottom portion 90 contains one or more icons that allow the patient 14 to, for example, access settings of the HCMA 34, access a care circle of support individuals, as will be discussed in greater detail below, upload information via the HCMA 34, and the like.

The HCMA 34 stores the HCO pages received from the HCS computing device 18-2, and the timestamps relating to such pages in an HCO cache 78-2. The HCMA 34 and the HCS computing device 18-2 can then subsequently exchange timestamp information to ascertain if new HCO pages exist on the HCS computing device 18-2 that have not been provided to the HCMA 34, as discussed above with regard to the HCS computing device 18-1.

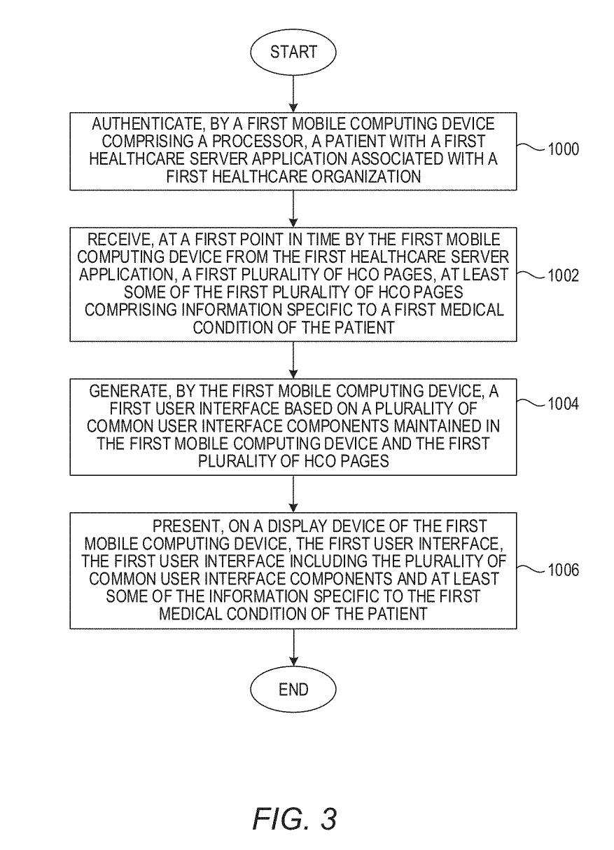

FIG. 3 is a flowchart of a method for facilitating patient-customized healthcare services from multiple healthcare organizations via a single healthcare application according to one embodiment. FIG. 3 will be discussed in conjunction with FIGS. 1 and 2. Preliminarily, it should be noted, that because the HCMA 34 is a component of the mobile computing device 12, functionality implemented by the HCMA 34 may be attributed herein to the mobile computing device 12 generally. Moreover, in examples where the HCMA 34 comprises software instructions that program the processor device 24 to carry out functionality discussed herein, functionality implemented by the HCMA 34 may be attributed herein to the processor device 24. Similarly, because the HCSA 58-1 is a component of the HCS computing device 18-1, functionality implemented by the HCSA 58-1 may be attributed herein to the HCS computing device 18-1 generally. Moreover, in examples where the HCSA 58-1 comprises software instructions that program the processor device 20-1 to carry out functionality discussed herein, functionality implemented by the HCSA 58-1 may be attributed herein to the processor device 20-1.

Referring now to FIG. 3, the mobile computing device 12 authenticates the patient 14 with the HCSA 58-1 associated with the first HCO 16-1 (FIG. 3, block 1000). The mobile computing device 12 receives a first plurality of HCO pages from the first HCSA 58-1, at least some of the first plurality of HCO pages comprising information specific to a first medical condition of the patient 14, in this example, the medical condition of diabetes (FIG. 3, block 1002). The mobile computing device 12 generates the user interface 68 based on the plurality of common user interface and framework components 48 maintained in the mobile computing device 12 and the first plurality of HCO pages (FIG. 3, block 1004). The mobile computing device 12 presents the user interface 68 on the display device 30. The user interface 68 includes the plurality of common user interface and framework components 48 and at least some of the information specific to the first medical condition of the patient 14 (FIG. 3, block 1006).

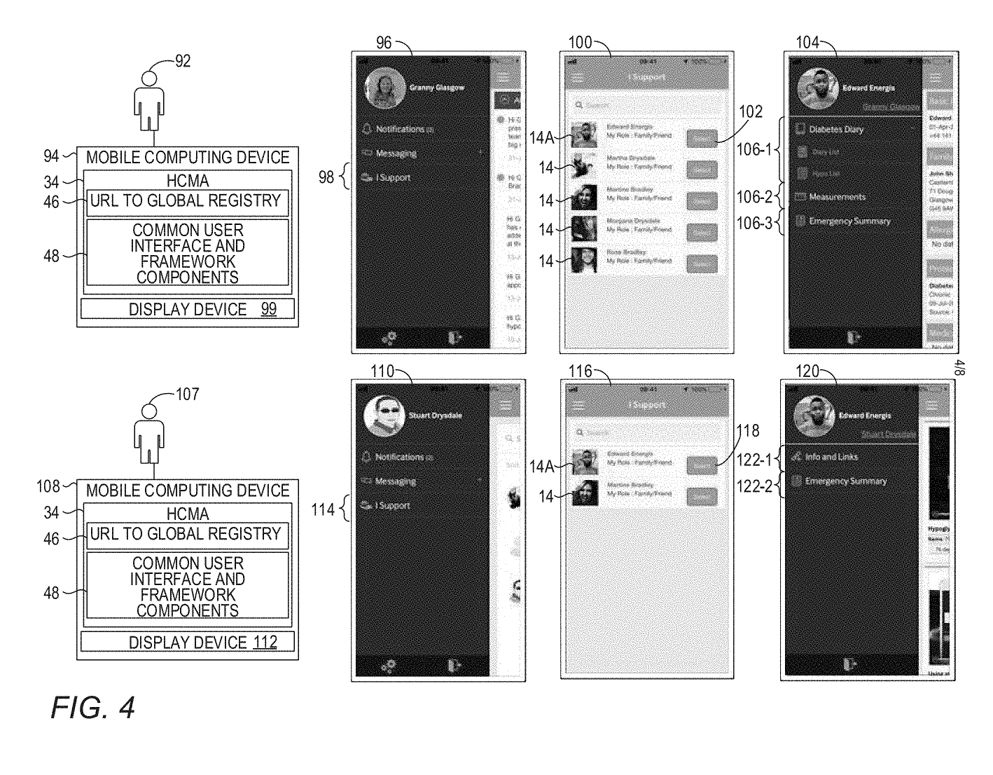

FIG. 4 is a block diagram illustrating additional aspects of the healthcare system according to some embodiments. Individuals registered in the global registry 42 of the healthcare system disclosed herein can be identified as having a role other than that of patient. In particular, a registered individual can be identified as being a member of a care circle who has authorized access to all or a portion of a patient's medical information maintained by the healthcare system. A member of a care circle will be referred to herein as a care user. A patient can designate what portions of the patient's medical information each care user can access. A care user, for example, may be a friend, family member, a neighbor, or the like. Referring again to FIG. 1, the patient 14 may indicate to the HCMA 34 that the patient 14 would like to designate another user of the healthcare system as a care user. If the individual is not yet identified in the global registry 42, the individual may first register with the global registry 42. The patient 14 may then designate via the HCMA 34 particular permissions for each designated care user that identify a subset of medical information regarding the patient 14 that can be provided to the care user. The patient 14 may designate different permissions for different care users. A care user may be designated as being a care user for multiple different patients 14, and may have different permissions for each patient 14.

Referring now to FIG. 4, a care user 92 has downloaded the HCMA 34 onto a mobile computing device 94 in a manner similar to that discussed with regard to the patient 14 in FIG. 1. While not illustrated for purposes of simplicity, the mobile computing device 94 includes a processor device and a memory. The care user 92 authenticates with the HCS computing device 18-1. The HCMA 34 presents a user interface 96 on a display device 99 of the mobile computing device 94. In this example, the user interface 96 indicates that the name of the care user 92 is Granny Glasgow. The user interface 96 includes a selectable control 98 labeled “I Support” that, upon selection by the care user 92, causes the HCMA 34 to generate and display the user interface 100. The user interface 100 presents a plurality of selectable controls, each of which corresponds to a patient 14 for which the care user 92 has been designated as a member of a care circle of the respective patient 14.

In this example, the care user 92 has been designated as a care user for five different patients 14. For purposes of illustration, assume that the care user 92 selects a selectable control 102 that corresponds to a patient 14A named Edward Energis. The HCMA 34 sends a message to the HCS computing device 18-1 that identifies the care user 92 and the patient 14A. The HCS computing device 18-1 accesses the user information 62 that corresponds to the patient 14A, and determines from the user information 62 those portions of the user information 62 that the patient 14A has designated can be viewed by the care user 92. The HCS computing device 18-1 generates a subset of HCO pages based on these privileges, and sends the HCO pages to the HCMA 34. Based on the subset of HCO pages and the common user interface and framework components 48 the HCMA 34 generates a user interface 104.

Note that the user interface 104 contains the same common components, such as background colors, font type, font size, and the like as the user interfaces 68 and 82 illustrated in FIG. 2. In this example, the patient 14A has designated that the care user 92 is permitted to access three classes of medical information of the patient 14A. Specifically, the care user 92 can view a “Diabetes Diary” identified via a selectable control 106-1, physiological measurements of the patient 14A identified via a selectable control 106-2, and an emergency summary designated by a selectable control 106-3. Upon selection by the care user 92 of any of the selectable controls 106, the HCMA 34 will present on the display device 99 additional detailed information regarding such class of medical information.

As another example, another care user 107 of the patient 14A has downloaded the HCMA 34 onto a mobile computing device 108 in a manner similar to that discussed with regard to the patient 14 in FIG. 1. While not illustrated for purposes of simplicity, the mobile computing device 108 includes a processor device and a memory. The care user 107 authenticates with the HCS computing device 18-1. The HCMA 34 presents a user interface 110 on a display device 112 of the mobile computing device 108. In this example, the user interface 110 indicates that the name of the care user 107 is Stuart Drysdale. The user interface 110 includes a selectable control 114 labeled “I Support” that, upon selection by the care user 107, causes the HCMA 34 to generate and display the user interface 116. The user interface 116 presents a plurality of selectable controls, each of which corresponds to a patient 14 for which the care user 107 has been designated as a member of a care circle of the respective patient 14.

In this example, the care user 107 has been designated as a care user for two different patients 14. For purposes of illustration assume that the care user 107 selects a selectable control 118 that corresponds to the patient 14A named Edward Energis. The HCMA 34 sends a message to the HCS computing device 18-1 that identifies the care user 107 and the patient 14A. The HCS computing device 18-1 accesses the user information 62 that corresponds to the patient 14A, and determines from the user information 62 those portions of the user information 62 that the patient 14A has designated can be viewed by the care user 107. The HCS computing device 18-1 generates a subset of HCO pages based on these privileges, and sends the HCO pages to the HCMA 34. Based on the subset of HCO pages and the common user interface and framework components 48, the HCMA 34 generates a user interface 120.

Note that the user interface 104 contains the same common components, such as background colors, font type, font size, and the like as the user interfaces 68 and 82 illustrated in FIG. 2. In this example, the patient 14A has designated that the care user 107 is permitted to access two classes of medical information of the patient 14A. Specifically, the care user 107 can view an “Info and Links” page identified via a selectable control 122-1 and an “Emergency Summary” identified via a selectable control 122-2. Note that the medical information that the care user 107 can view is different from the medical information that the care user 92 can view because the patient 14A has designated that the care user 92 and the care user 107 have different privileges that allow them to see different portions of the patient 14A's medical information.

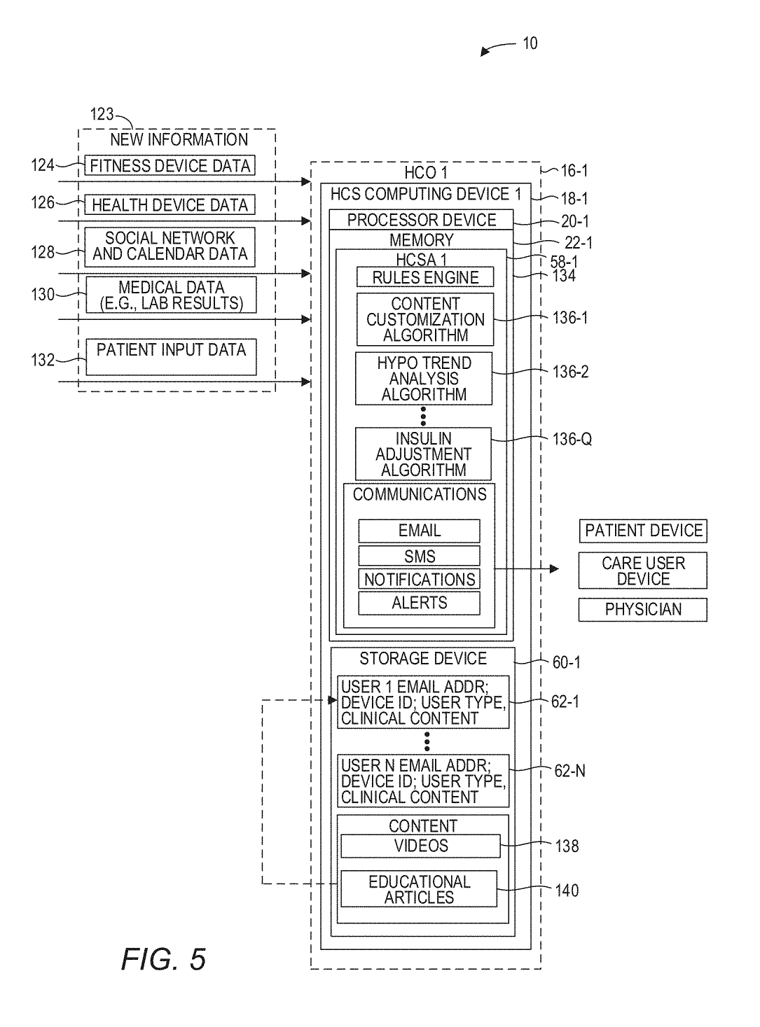

FIG. 5 is a block diagram of a portion of the environment 10 suitable for illustrating additional aspects of the healthcare system disclosed herein according to some embodiments. In particular, the HCSA 58-1 continuously, over time, reacts and performs actions in response to new information 123 received about the patient 14. The new information 123 may be received by the HCSA 58-1 via any of a number of different paths. For example, the mobile computing device 12 can be coupled to a fitness device such as a Fitbit that generates fitness device data 124 regarding activities of the patient 14. The information can be provided to the HCSA 58-1 via the HCMA 34 on the mobile computing device 12 automatically, or via a website associated with the fitness device. This information 123 can be stored in the user information 62-1. Similarly, the HCSA 58-1 may receive health device data 126 that identifies physiological measurements of the patient 14, such as, by way of non-limiting example, glucose levels, blood pressure, pulse, and the like. Again, the health device data 126 may be provided by the mobile computing device 12, or via a website associated with the particular health measurement device, and stored by the HCSA 58-1 in the user information 62-1.

In some embodiments, the patient 14 may provide access by the HCMA 34 to social network feeds and calendars of the patient 14. The HCMA 34 may periodically provide such social network and calendar data 128 to the HCSA 58-1. Physicians and medical personnel may generate medical data 130, such as lab results and the like, that may be automatically or manually entered into the HCSA 58-1 and stored in the user information 62-1. The HCMA 34 may periodically receive from the HCSA 58-1 questionnaires and/or assessments that are presented to the patient 14 from time to time to solicit information from the patient 14. Patient input data 132 provided by the patient 14 in response to such questionnaires and assessments is provided by the HCMA 34 to the HCSA 58-1.

When such new information 123 is received by the HCSA 58-1, the HCSA 58-1 may generate timestamps and store such new information 123 in the user information 62-1 in conjunction with such timestamps so that the HCSA 58-1 can maintain track of what information in the user information 62-1 is new information 123, and thus hasn't been previously processed. Periodically, intermittently, upon demand, or in response to particular information, a rules engine 134 processes the user information 62-1 analyzing the new information 123 received by the HCSA 58-1. The rules engine 134 may operate in conjunction with one or more algorithms 136-1-136-Q to determine whether any new information 123 received by the HCSA 58-1 triggers an action. The action may comprise any number of different things, including, by way of non-limiting example, the addition of educational content to the user information 62-1 for subsequent presentation to the patient 14, the modification of a current procedure followed by the patient 14, the generation of a communication such as an email, an SMS message, notifications and/or alerts to one or more of the patient 14, a care user of the patient 14, or a physician of the patient 14.

As an example, a content customization algorithm 136-1 may, in response to the receipt of new information 123 by the HCSA 58-1, select one or more videos 138, and/or one or more educational articles 140, and content in the user information 62-1 such that the content will be downloaded to the HCMA 34 for presentation to the patient 14. As an example, the rules engine 134 may include a rule that the systolic blood pressure of the patient 14 should not exceed 140 mmHg. The health device data 126 may include a systolic blood pressure reading of 150 mmHg. In response to the new health device data 126, the rules engine 134 in conjunction with the content customization algorithm 136-1 may select educational articles 140 that relate to activities the patient 14 can do to lower blood pressure. The next time the HCMA 34 connects to the HCSA 58-1, such educational articles 140 will be downloaded to the HCMA 34 for presentation to the patient 14.

As another example, the rules engine 134 in conjunction with an insulin adjustment algorithm 136-3 may analyze the new information 123 received, such as new medical data 130, that identifies an insulin level of the patient 14. The insulin adjustment algorithm 136-3, based on the insulin level indicated in the medical data 130, may modify the user information 62-1 to adjust an amount of insulin injected by the patient 14.

In another example, the new information 123 received by the HCSA 58-1 may include some physiological measurement, such as blood pressure, blood glucose level, or the like. The rules engine 134 may include a rule that if such physiologic measurement is above a predetermined threshold, the HCSA 58-1 should immediately generate an SMS text message alert warning about the high physiological measurement and send the alert to the mobile computing device 12. The rule may also indicate that the alert should be sent to one or more care users of the patient 14. Based on the care privileges identified in the user information 62-1, some care users of the patient 14 may receive the alert, and other care users of patient 14 may not.

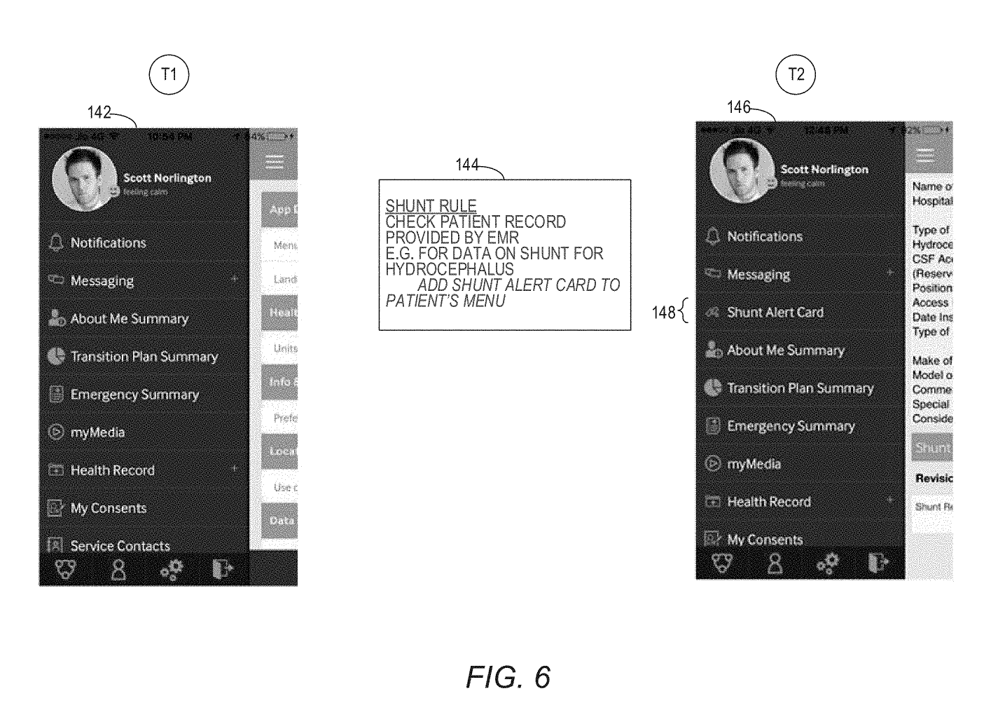

FIG. 6 is a diagram illustrating how the HCSA 58-1 may update user information 62 of a patient 14 in response to the receipt of new information 123 associated with the patient 14 according to one embodiment. At a time T1, the HCMA 34 may present the patient 14 who has the medical condition spina bifida with the user interface 142. Subsequent to time T1, the HCSA 58-1 receives a medical record that indicates the patient 14 has received a shunt. The rules engine 134 includes a rule 144 that indicates if the user information 62 of the patient 14 indicates that the patient 14 has received a shunt, then a shunt alert card should be added to the user information 62. The rules engine 134 determines, based on the new information 123, that the patient 14 has received a shunt and, in response, generates a shunt alert card and stores the shunt alert card in the user information 62 associated with the patient 14. At a time T2, the HCMA 34 accesses the HCSA 58-1, and is provided the new shunt alert card. The HCMA 34 presents the user interface 146 which now includes a selectable control 148 that indicates the patient 14 has a shunt alert card.

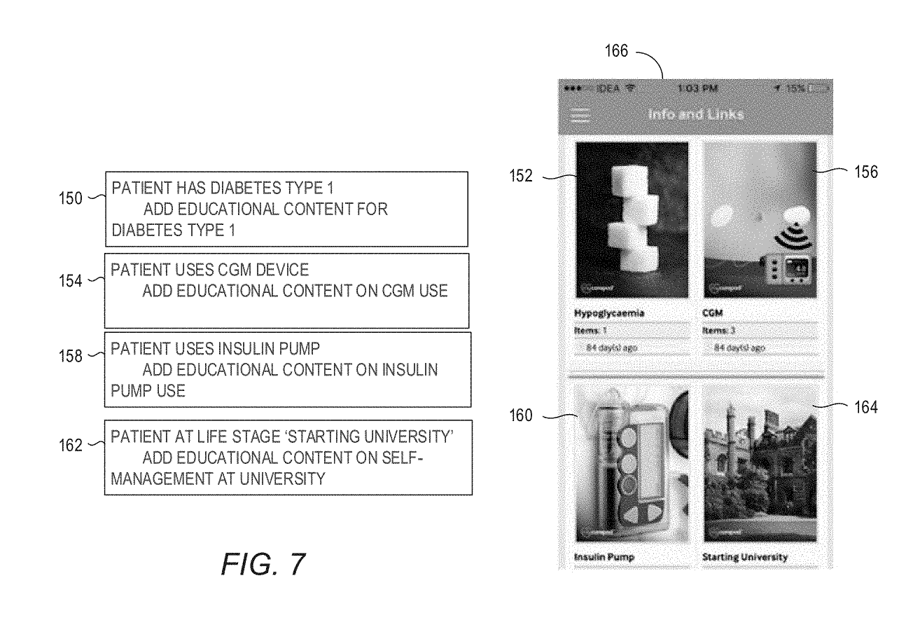

FIG. 7 is a diagram illustrating how the HCSA 58-1 may update user information 62 of a patient 14 in response to the receipt of new information 123 associated with the patient 14 according to another embodiment. In this example, the new information 123 dictates that the patient 14 has diabetes type I, uses a Continuous Glucose Monitoring (CGM) device, uses an insulin pump, and has indicated that they are at a “starting university” life stage. The rules engine 134 includes a rule 150 that indicates that if the patient 14 has diabetes type I, educational content relating to diabetes type I should be added to the user information 62 of the patient 14. The rules engine 134 in conjunction with the content customization algorithm 136-1 selects an article 152 relating to hypoglycemia and inserts the article 152, or a link thereto, in the user information 62 of the patient 14.

The rules engine 134 includes a rule 154 that indicates that if the patient 14 uses a CGM device, educational content relating to the use of a CGM device diabetes type I should be added to the user information 62 of the patient 14. The rules engine 134 in conjunction with the content customization algorithm 136-1 selects an article 156 relating to the use of a CGM device and inserts the article 156, or a link thereto, in the information 62 of the patient 14.

The rules engine 134 includes a rule 158 that indicates that if the patient 14 uses an insulin pump, educational content relating to the use of an insulin pump should be added to the user information 62 of the patient 14. The rules engine 134 in conjunction with the content customization algorithm 136-1 selects an article 160 relating to the use of an insulin pump and inserts the article 160, or a link thereto, in the information 62 of the patient 14.

The rules engine 134 includes a rule 162 that indicates that if the patient 14 is at the “starting university” life stage, educational content relating to the “starting university” life stage should be added to the user information 62 of the patient 14. The rules engine 134 in conjunction with the content customization algorithm 136-1 selects an article 164 relating to the “starting university” life stage and inserts the article 160, or a link thereto, in the information 62 of the patient 14.

The patient 14 may then initiate the HCMA 34. The HCMA 34 accesses the HCSA 58-1, and downloads the articles inserted into the user information 62 of the patient 14 by the rules engine 134. The HCMA 34 may then present on the mobile computing device of the patient 14 a user interface 166 that identifies the articles 152, 156, 160, and 164, and allows the patient 14 to read the articles 152, 156, 160, and 164.

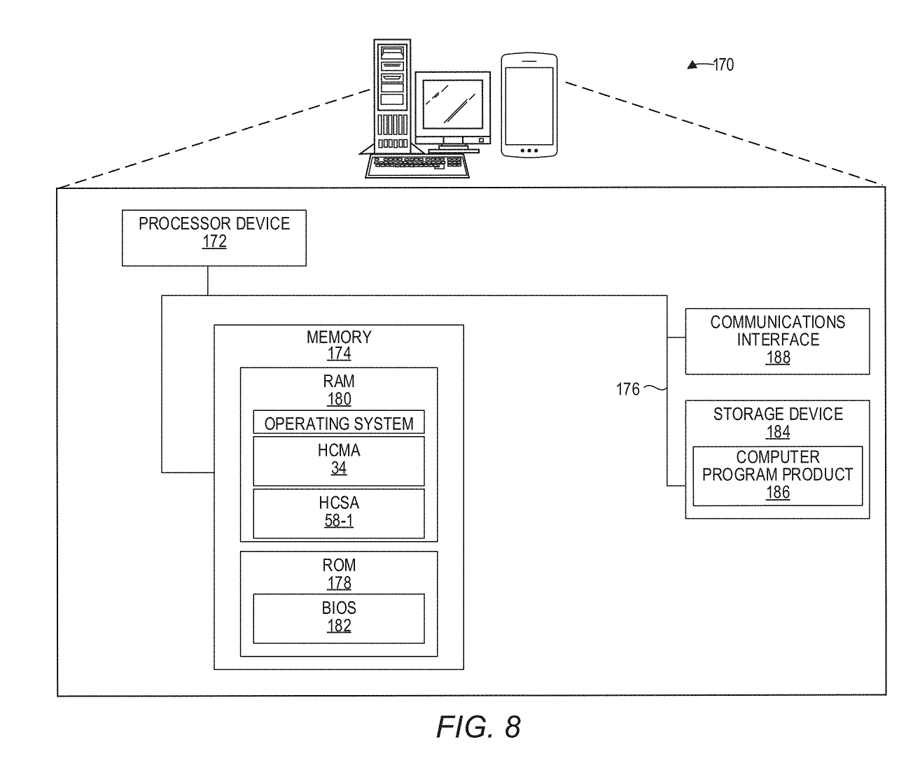

FIG. 8 is a block diagram of a computing device 170 suitable for implementing any of the computing devices discussed herein, including, the mobile computing device 12, the HCS computing device 18-1, the HCS computing device 18-2, the computing device 36, and the like. The computing device 170 may comprise any computing or electronic device capable of including firmware, hardware, and/or executing software instructions to implement the functionality described herein, such as a computer server, a desktop computing device, a laptop computing device, a smart phone, a computing tablet, or the like. The computing device 170 includes a processor device 172, a memory 174, and a system bus 176. The system bus 176 provides an interface for system components including, but not limited to, the memory 174 and the processor device 172. The processor device 172 can be any commercially available or proprietary processor.

The system bus 176 may be any of several types of bus structures that may further interconnect to a memory bus (with or without a memory controller), a peripheral bus, and/or a local bus using any of a variety of commercially available bus architectures. The memory 174 may include non-volatile memory 178 (e.g., read-only memory (ROM), erasable programmable read-only memory (EPROM), electrically erasable programmable read-only memory (EEPROM), etc.), and volatile memory 180 (e.g., random-access memory (RAM)). A basic input/output system (BIOS) 182 may be stored in the non-volatile memory 178 and can include the basic routines that help to transfer information between elements within the computing device 170. The volatile memory 180 may also include a high-speed RAM, such as static RAM, for caching data.

The computing device 170 may further include or be coupled to a non-transitory computer-readable storage medium such as a storage device 184, which may comprise, for example, an internal or external hard disk drive (HDD) (e.g., enhanced integrated drive electronics (EIDE) or serial advanced technology attachment (SATA)), HDD (e.g., EIDE or SATA) for storage, flash memory, or the like. The storage device 184 and other drives associated with computer-readable media and computer-usable media may provide non-volatile storage of data, data structures, computer-executable instructions, and the like. Although the description of computer-readable media above refers to an HDD, it should be appreciated that other types of media that are readable by a computer, such as Zip disks, magnetic cassettes, flash memory cards, cartridges, and the like, may also be used in the operating environment, and, further, that any such media may contain computer-executable instructions for performing novel methods of the disclosed examples.

A number of modules can be stored in the storage device 184 and in the volatile memory 180, including the HCMA 34 and/or the HCSA 58-1, which may implement the functionality described herein in whole or in part. All or a portion of the examples may be implemented as a computer program product 186 stored on a transitory or non-transitory computer-usable or computer-readable storage medium, such as the storage device 184, which includes complex programming instructions, such as complex computer-readable program code, to cause the processor device 172 to carry out the steps described herein. Thus, the computer-readable program code can comprise software instructions for implementing the functionality of the examples described herein when executed on the processor device 172. The processor device 172, in conjunction with the HCMA 34 and/or the HCSA 58-1 in the volatile memory 180, may serve as a controller, or control system, for the computing device 170 that is to implement the functionality described herein. The computing device 170 may also include a communications interface 188 suitable for communicating with a network as appropriate or desired.

Those skilled in the art will recognize improvements and modifications to the preferred embodiments of the disclosure. All articles improvements and modifications are considered within the scope of the concepts disclosed herein and the claims that follow.

What is claimed is:

1. A method comprising:
authenticating, by a first mobile computing device comprising a processor, a patient with a first healthcare server application associated with a first healthcare organization;
receiving, at a first point in time by the first mobile computing device from the first healthcare server application, a first plurality of healthcare organization (HCO) pages, at least some of the first plurality of HCO pages comprising information specific to a first medical condition of the patient;
generating, by the first mobile computing device, a first user interface based on a plurality of common user interface components maintained in the first mobile computing device and the first plurality of HCO pages; and
presenting, on a display device of the first mobile computing device, the first user interface, the first user interface including the plurality of common user interface components and at least some of the information specific to the first medical condition of the patient.

2. The method of claim 1 further comprising:
authenticating, by the first mobile computing device, the patient with a second healthcare server application associated with a second healthcare organization that is different from the first healthcare organization;
receiving, by the first mobile computing device from the second healthcare server application, a second plurality of HCO pages, at least some of the second HCO pages comprising information specific to a second medical condition of the patient;
generating, by the first mobile computing device, a second user interface based on the plurality of common user interface components maintained in the first mobile computing device and the second plurality of HCO pages; and
presenting, on the display device, the second user interface, the second user interface including the plurality of common user interface components and at least some of the information specific to the second medical condition of the patient.

3. The method of claim 1 further comprising:
receiving, by the first mobile computing device, input from the patient that contains data identifying a characteristic of the patient;
sending, by the first mobile computing device, the input to the first healthcare server application;
receiving, by the first mobile computing device from the first healthcare server application, in response to sending the input, a first educational content that relates to the first medical condition; and
presenting, by the first mobile computing device on the display device, the first educational content.

4. The method of claim 1 further comprising:
prior to authenticating the patient with the first healthcare server application, authenticating the patient with a global registry;
receiving, from the global registry, a first healthcare organization address that identifies an electronic address of the first healthcare server application, and a second healthcare organization address that identifies an electronic address of the second healthcare server application;
presenting, on the display device, a first healthcare organization reference that refers to the first healthcare organization and a second healthcare organization reference that refers to the second healthcare organization;
receiving, via the display device and from the patient, input that selects the first healthcare organization reference; and
in response to receiving the input that selects the first healthcare organization reference, authenticating the patient with the first healthcare server application associated with the first healthcare organization.

5. The method of claim 1 wherein the first plurality of HCO pages comprises one or more of previous physiological measurements of the patient, medications prescribed to the patient for the first medical condition, laboratory reports of the patient, and educational materials relating to the first medical condition of the patient.

6. The method of claim 1 further comprising:
authenticating, by a second mobile computing device comprising a processor with the first healthcare server application, a first care user that has been designated in a care circle of the patient; and
receiving, by the second mobile computing device from the first healthcare server application, a subset of the first plurality of HCO pages based on privileges associated with the first care user stored on the first healthcare server application.

7. The method of claim 1 further comprising:
receiving, from an external device communicatively coupled to the first mobile computing device, physiologic data that quantifies a physiological condition of the patient;
sending, by the first mobile computing device to the first healthcare server application, the physiologic data;
receiving, by the first mobile computing device from the first healthcare server application, an alert identifying a warning based on the physiologic data; and
presenting the warning on the display device.

8. The method of claim 1 further comprising:
receiving, from an external device communicatively coupled to the first mobile computing device, physiologic data that quantifies a physiological condition of the patient;
sending, by the first mobile computing device to the first healthcare server application, the physiologic data;
receiving, by the first mobile computing device from the first healthcare server application, educational material based on the physiologic data; and
presenting the educational material on the display device.

9. A mobile computing device comprising:
a display device;
a memory; and
a processor device coupled to the memory and configured to:
authenticate a patient with a first healthcare server application associated with a first healthcare organization;
receive, from the first healthcare server application, a first plurality of healthcare organization (HCO) pages, at least some of the first plurality of HCO pages comprising information specific to a first medical condition of the patient;
generate a first user interface based on a plurality of common user interface components maintained in the mobile computing device and the first plurality of HCO pages; and
present, on the display device, the first user interface, the first user interface including the plurality of common user interface components and at least some of the information specific to the first medical condition of the patient.

10. The mobile computing device of claim 9, wherein the processor device is further configured to:
authenticate the patient with a second healthcare server application associated with a second healthcare organization that is different from the first healthcare organization;
receive, from the second healthcare server application, a second plurality of HCO pages, at least some of the second plurality of HCO pages comprising information specific to a second medical condition of the patient;
generate a second user interface based on the plurality of common user interface components maintained in the mobile computing device and the second plurality of HCO pages; and
present, on the display device, the second user interface, the second user interface including the plurality of common user interface components and at least some of the information specific to the second medical condition of the patient.

11. The mobile computing device of claim 9, wherein the processor device is further configured to:
receive, from an external device communicatively coupled to the mobile computing device, physiologic data that quantifies a physiological condition of the patient;
send, to the first healthcare server application, the physiologic data;
receive, from the first healthcare server application, an alert identifying a warning based on the physiologic data; and
present the warning on the display device.

12. The mobile computing device of claim 9, wherein the processor device is further configured to:
receive, from an external device communicatively coupled to the mobile computing device, physiologic data that quantifies a physiological condition of the patient;
send, to the first healthcare server application, the physiologic data;
receive, from the first healthcare server application, educational material based on the physiologic data; and
present the educational material on the display device.

13. A method comprising:
receiving, by a healthcare server application from a first mobile computing device, authentication information that authenticates a first care user with the healthcare server application, the healthcare server application being associated with a first healthcare organization, the first care user being identified as a care user of a patient of a plurality of patients of the first healthcare organization, the healthcare server application comprising patient healthcare records associated with a plurality of different patients;
receiving, from the first mobile computing device, a request for healthcare information associated with the patient;
accessing care user privilege information associated with the patient, the care user privilege information identifying a first subset of healthcare information associated with the patient that may be provided to the first care user;
generating content based on the first subset of healthcare information; and
sending the content to the first mobile computing device.

14. The method of claim 13 further comprising:
receiving, by the healthcare server application from a second mobile computing device, authentication information that authenticates a second care user with the healthcare server application, the second care user being identified as a care user of the patient;
receiving, from the second mobile computing device, a request for healthcare information associated with the patient;
accessing the care user privilege information associated with the patient, the care user privilege information identifying a second subset of healthcare information associated with the patient, which is different from the first subset of healthcare information, that may be provided to the first care user;
generating content based on the second subset of healthcare information; and
sending the content to the second mobile computing device.

15. The method of claim 14 further comprising:
receive, from an external computing device, laboratory information that identifies physiologic information associated with the patient;
generate content that includes at least some of the laboratory information;
access the care user privilege information associated with the patient; and
based on the care user privilege information, send the content to the first mobile computing device, and inhibit sending the content to the second mobile computing device.

16. The method of claim 14 further comprising:
receive, from an external computing device, laboratory information that identifies physiologic information associated with the patient;
access a plurality of conditions;
determine that the laboratory information contains a physiologic measurement that is outside of a desired range based on one of the plurality of conditions;
based on determining that the physiologic measurement is outside of the desired range, generate an alert that contains information that indicates that the physiologic measurement is outside of the desired range; and
based on the care user privilege information, send the alert to the first mobile computing device and inhibit sending the alert to the second mobile computing device.

17. A system comprising:
a mobile computing device comprising:
a display device;
a memory; and
a mobile processor device coupled to the memory; and
a healthcare server computing device comprising a memory;
wherein the mobile processor device is configured to:
authenticate a patient with the healthcare server computing device, the healthcare server computing device being associated with a first healthcare organization;
receive, from the healthcare server computing device, a first plurality of HCO pages, at least some of the first plurality of healthcare organization (HCO) pages comprising information specific to a first medical condition of the patient;
generate a first user interface based on a plurality of common user interface components maintained in the mobile computing device and the first plurality of HCO pages; and
present, on the display device, the first user interface, the first user interface including the plurality of common user interface components and at least some of the information specific to the first medical condition of the patient.

18. The system of claim 17 wherein the healthcare server computing device comprises a server processor device, the server processor device being configured to:
receive physiological data associated with a user that corresponds to the mobile computing device;
access a rule that identifies acceptable values for the physiological data;
determine that the physiological data is outside of the acceptable values;
in response to determining that the physiological data is outside of the acceptable values, select content that contains information relating to keeping the physiological data within the acceptable values;
automatically send the content to the mobile computing device.

19. The system of claim 17 wherein the healthcare server computing device comprises a server processor device, server processor device being configured to:
receive physiological data associated with a user that corresponds to the mobile computing device;
access a rule that identifies acceptable values for the physiological data;
determine that the physiological data is outside of the acceptable values;
in response to determining that the physiological data is outside of the acceptable values, generate an alert and send the alert to the mobile computing device.
